(19) United States
(12) Patent Application Publication
(10) Pub. No.: US 2020/0026099 A1
(43) Pub. Date: Jan. 23, 2020

(54) A CONTROL SYSTEM FOR A SWITCHABLE LENS

(71) Applicant: DYNAMIC VISION SYSTEMS LIMITED, Leed (GB)

(72) Inventors: John Clifford Jones, Greater Manchester (GB); Helen Frances Gleeson, Greater Manchester (GB); Philip Bruce Morgan, Greater Manchester (GB)

(21) Appl. No.: 16/471,117

(22) PCT Filed: Dec. 14, 2017
(86) PCT No.: PCT/GB2017/053751

§ 371 Date: Jun. 19, 2019

(30) Foreign Application Priority Data
Dec. 19, 2016 (GB) 1621571.7

(51) Int. Cl. G02C 7/08; A61B 5/00; G02C 7/04

(52) U.S. Cl. CPC G02C 7/083; G02C 7/04; A61B 5/6821

(57) ABSTRACT

Disclosed is a dynamically switchable contact lens, operable to switch between first and second focal states, each focal state having a different optical property, the lens comprising: a power source; a controller, operable to control the operation of the lens; a transceiver operable to communicate with a second lens and/or an external controller; wherein the controller is operable to pair the lens with the second lens by means of the external controller.

The present invention relates to switchable lens devices, and in particular switchable contact lenses and a system for controlling such lenses. In particular, the system comprises means for controlling the focal state using radio signals, for charging a lens comprising a charge storage device by radio signal (such as Bluetooth Low Energy (BLE), RFID and/or NFC), and for pairing two lenses to react simultaneously when triggered by an appropriate control signal. Throughout this specification, focal state represents an operational state of the contact lens, whereby it is configured in a first focal state to have a first optical property and in a second focal state to have a second optical property.

Vision correction for conditions such as presbyopia has been the subject for switchable lenses for several decades. A variety of forms of operation have been proposed, including switchable diffractive elements and switchable liquid crystals devices with curved substrates.

Suitable devices use electrode structures deposited onto the internal surfaces of a cavity or inserted within the lens that contains the electro-optic medium. When a field is applied across the medium, it causes a change in refractive index, and hence alters the focussing power of the lens. Embodiments of the present invention utilise an electro-refractive medium that draws minimal current whilst activated and which suitably operates with voltages of less than 10V and, preferably, less than 5V. For these reasons, the electro-refractive medium may be a thin layer of liquid crystal, such as a nematic, cholesteric, blue-phase or ferroelectric liquid crystal, although lenses made from other electro-refractive materials, such as electro-wetting liquids, electrophoretic dispersions, dielectro-phoretic liquids, Kerr effect or Pockels effect media may be suitable.

Embodiments of the present invention are primarily concerned with the control system for use with switchable lenses, rather than the optical properties of the switchable lenses.

It is an aim of embodiments of the present invention to provide an electronic control system for use with switchable lenses, irrespective of the optical or refractive properties relied upon to implement the switchable lenses.

According to the present invention there is provided an apparatus and method as set forth in the appended claims. Other features of the invention will be apparent from the dependent claims, and the description which follows.

Embodiments of the invention may take one of several forms. In a first form, a pair of lenses is provided, which are operable, in normal use, to switch focal state as a pair, independent of any external controller. In a second form, a pair of lenses is provided, which are operable to switch focal state as a pair, together with an external controller. Furthermore, either first or second forms may be further provided with a further processing device and/or a charging device, which may also provide a cleaning function, whereby the associated lenses are deposited in the device at the end of a period of use and are cleaned whilst the lens internal power source may be recharged.

Embodiments of the present invention provide an operating system for contact lenses. The system provides wireless power and/or triggering for switching between different focal points. The system comprises a control function, formed from electronic components that are mounted on to or internal to the lens, or which may include further electronic components mounted into a control unit, separate from and external to the lens. The lens includes means for communicating between the control unit and the lens. When mounted within a lens, the control unit is in communication with the control unit of a second paired lens, so that simultaneous, or near simultaneous, changes in focal power can be obtained for the lens pair.

In the case where the control unit is mounted in a separate unit, it uses radio communication with both paired lenses. Both lenses have individual identification codes, such as RFID. The control unit, whether mounted within one of the lenses or in an external unit, sends a ‘transmit’ signal that includes the two ID codes of the paired lenses (and potentially a voltage level, or similar control information to indicate the focussing level), and listens for a ‘receipt’ signal. If a lens receives an incorrect ID code, it remains in its current state. If either lens recognises its own ID code, it transmits a ‘ready’ signal. Once the control unit has received the ‘ready’ signal from both lenses in a pair, it transmits a ‘trigger’ signal to the lenses, causing them to change the applied voltage to the appropriate level. In a second embodiment, the lenses also reply with a second ‘receipt’ signal. If the control unit does not receive two such signals, it may repeat the process.

For a better understanding of the invention, and to show how embodiments of the same may be carried into effect, reference will now be made, by way of example, to the accompanying diagrammatic drawings in which:

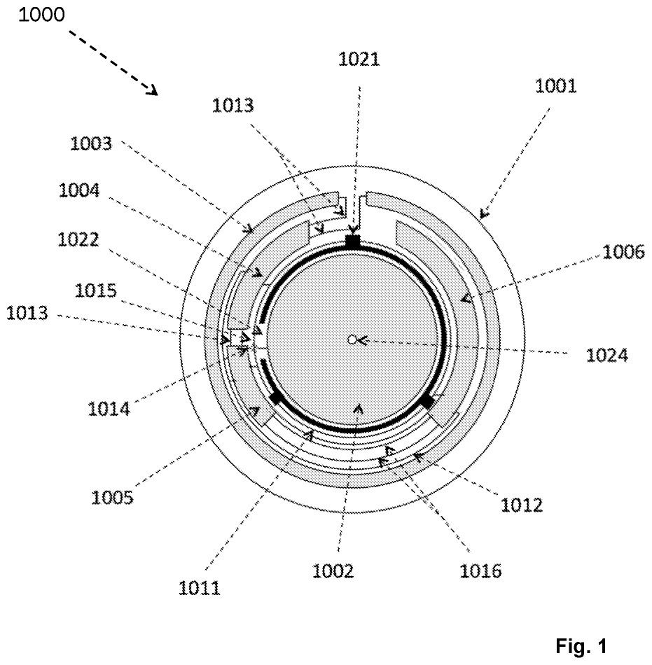

FIG. 1 shows a lens according to a first embodiment of the present invention;

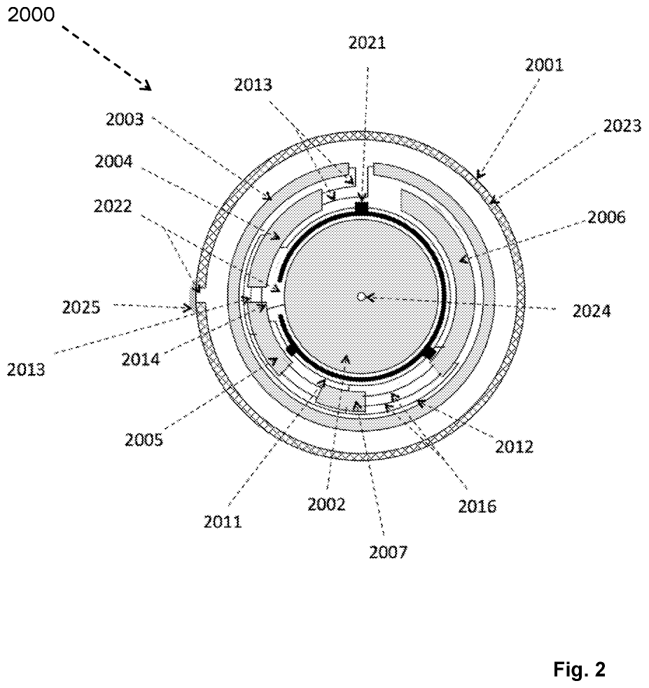

FIG. 2 shows lens according to a second embodiment of the present invention;

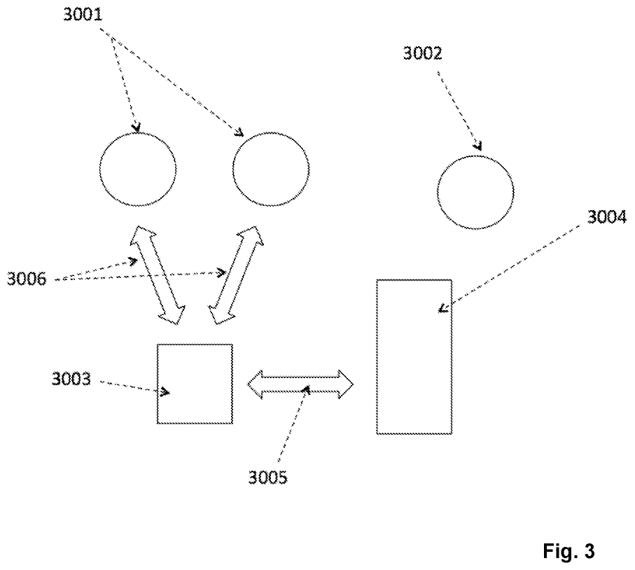

FIG. 3 shows a schematic of a control system and associated lenses according to an embodiment of the present invention;

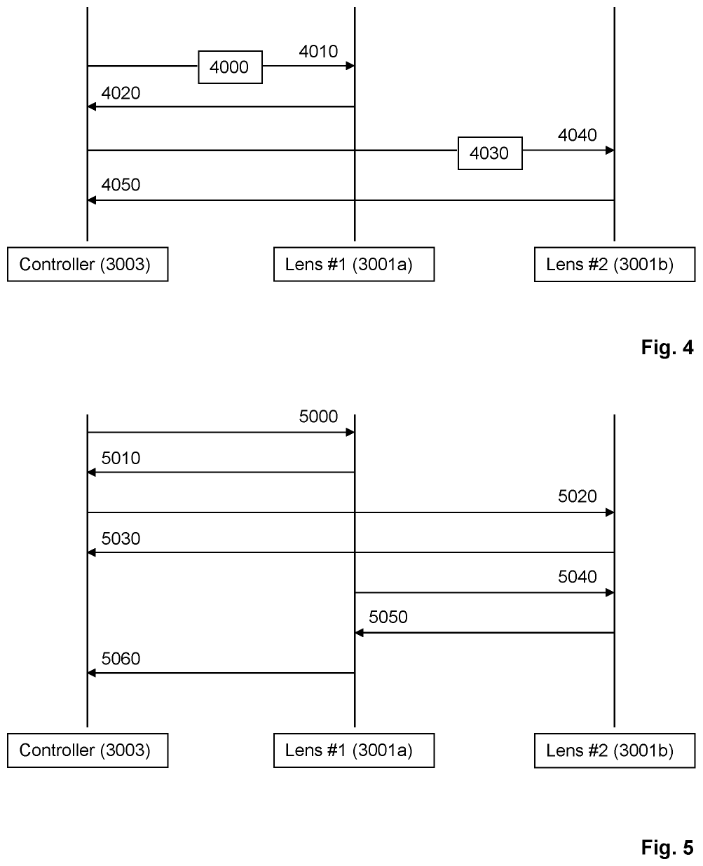

FIG. 4 shows a message exchange involved in a pairing operation according to an embodiment of the invention;

FIG. 5 shows a message exchange involved in a pairing operation according to another embodiment of the invention

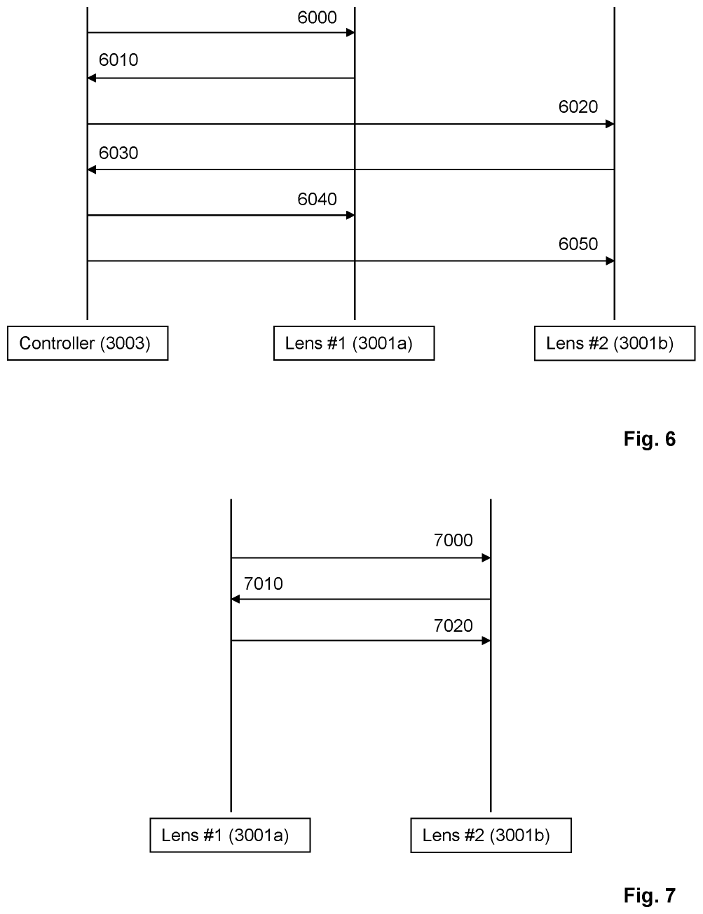

FIG. 6 shows a message exchange in an operational mode according to an embodiment of the invention;

FIG. 7 shows a message exchange in an operational mode according to another embodiment of the invention; and

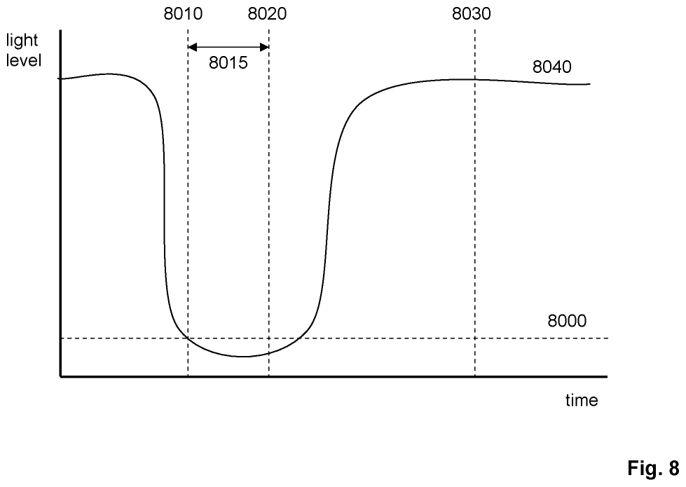

FIG. 8 illustrates how wink/blink length can be used to control operation of a lens according to an embodiment of the invention.

FIGS. 1 and 2 show a lens (1000, 2000) according to first and second embodiments of the invention. Similarly numbered features represent similar components or functions.

The bottom substrate (1001, 2001) of the contact lens has a curvature to approximately match the outer curvature of the anterior cornea of the human eye. It may have a non-uniform spacing and high refractive index to give vision correction properties, or alternatively, have a uniform spacing of the liquid crystal but means for providing a non-uniform electric field included. There is provided a lower electrode structure on the upper surface of the lens substrate (1002, 2002). The electrode may be circular with a diameter that covers the pupil under normal light conditions (approximately 4-5 mm), or slightly wider to cover low light levels (5-8 mm). The electrode may be formed from a transparent conductor such as Indium Tin Oxide (ITO), conducting nano-wires (e.g. silver, gold or CNT), conducting polymer such as Poly(3,4-ethylenedioxythiophene) (known as PEDOT), or graphene.

The surface of at least one substrate comprises an antenna (1003, 2003) for communication to (and/or from) the lens and, in at least one embodiment, for providing power, either momentarily to cause a change in focal state, or to charge an internal power source (1006, 2006) to be described. The antenna has a generally spiral structure, and is formed from either evaporated gold or silver, but may also be formed from printed gold nanowires or silver nanowires. The antenna has a resistance below 100 ohm, preferably below 50 ohms and most preferably below 10 ohm. It may be contained on any of the surfaces of the lens, and is not restricted to the upper surface of the lower substrate as shown, which is merely exemplary.

The lens system further comprises a receiver unit or transceiver (combined transmitter and receiver) unit (1004, 2004) that is connected to the antenna through conducting connectors (1013, 2013). These conducting connectors are typically formed from the same material as the electrode structure 1002, as are the other conductors on the substrate (1011, 1012, 1014, 1015 and 1016, 2011, 2012, 2014, 2015 and 2016). The receiver or transceiver is further connected to a microcontroller 1005, 2005, which acts to control the operations of the lens.

The substrate also comprises a microcontroller (1005, 2005). An example of a suitable microcontroller is the Atmel BTLC1000 system on chip (soc). This includes Bluetooth 4.1 (BLE) functionality, in addition to having 128 k RAM and 128 k ROM to accommodate the lens control software. Such a system on chip is approximately 2.1×2.4 mm in area. Further embodiments may utilize ASIC technology to further integrate and miniaturise the microcontroller function.

Both the receiver and the microcontroller may comprise elements formed from a high mobility semiconductor deposited onto the substrate (e.g. poly-silicon or Indium Gallium Zinc Oxide) or alternatively by a small silicon chip bonded to the substrate and connected to conductive tracks provided on the lens through gold-bump connectors. It is preferable that both the microcontroller and the transceiver are shielded from incident light using a light-blocking layer. Furthermore, it is preferable if this is on both sides of the substrate, to minimise light leakage both front and back from being incident on the semiconducting elements.

The electrodes, the transceiver/receiver (1004, 2004) and the microcontroller (1005, 2005) are powered by a charge storage unit (1006, 2006) formed from a capacitor, preferably a super capacitor, or a battery, preferably a re-chargeable battery. For example, a sugar-based rechargeable battery (such as one based on a synthetic enzymatic pathway is suitable). Such batteries can produce 300 Wh/kg, which is suitable for this application. The power source may be connected to the antenna via the receiver and/or microcontroller (and/or sensor unit 2007, to be described later) for re-charging via connectors (1016, 2016). Switching between charging and discharging (or lens powering) modes is managed by the microcontroller (1005, 2005) using firmware provided in the microcontroller (1005, 2005). The power supply (1006, 2006) supplies electrical power to the microcontroller (1005, 2005) and transceiver/receiver (1004, 2004) and sensor (2007) via electrical connectors (1011, 1012, 2011 and 2012).

In the embodiment shown in FIGS. 1 and 2, it is shown that the common electrode for the power supply is the inner connector (1011, 2012) since this readily makes contact with the 1D conductive seal (1024, 2024), and therefore to the earth electrode on the opposing lens substrate (not shown) that opposes the high voltage electrode (1002, 2002) and between which the electro-active element (e.g. liquid crystal) is situated. 1D, or one-dimensional, seals as known in the art, allow connections to be made between different parts of the lens in a single direction, typically from one layer to another, without signals being propagated transverse to the one dimension in question. Typically, such 1D seals utilise a plurality of discrete metallic (e.g. gold) particles arranged randomly within the seal but with a diameter that is at least equal to the spacing of the upper and lower electrodes to form a conductor between those electrodes but at a given density, allowing a signal to propagate via the column only and not from gold particle to gold particle. The conductive seal (1024, 2024) is formed from an ultra-violet (UV) or thermal adhesive, with added gold particles of the correct diameter and weight % concentration to form a 1D connection between electrodes on the upper and lower lens substrates, without giving substantial lateral conductivity. This adhesive layer may also comprise additional spherical or plastic spacers which have a lower diameter than the gold particles, but which set the correct spacing between the two lenses.

The conductive seal (1024, 2024) provides connection of the opposing electrode to the power supply (1006, 2006) and the opposing electrode, as well as providing a seal around the majority of the liquid crystal sample. Often, the liquid crystal will be introduced into the cavity between the electrodes via a filling hole in the (conducting) seal as shown in FIGS. 1 and 2 at 1022, 2022. The filling hole is also the point at which the connection is made from the microcontroller (1005, 2005) to the high voltage electrode (1002) via the connector 1014. An insulating film (1015) is deposited between the connector (1014) and the low voltage bus line (1011) to prevent shorting. An alternative arrangement is shown in FIG. 2, whereby the low voltage bus-line (2012) may be routed around the right-hand-side (as shown) of the inner structure to provide power to the receiver/transceiver unit (2004).

The system also comprises a central spacer (1024, 2024) to help maintain the correct spacing between the two lens substrates. This spacer should be made from an insulating adhesive and plastic/glass spacers, and be of minimal diameter (typically 0.2-0.4 mm). Alternatively, it may be formed from the lens material during the lathing or moulding process used to form one of the composite lenses. A second seal (2023) may also be used to ensure that the substrates remain correctly spaced and attached to each other. The liquid crystal may be introduced into the lens cavity either by printing the correct amount of material before sealing the substrates together, or by introducing through capillary action (or vacuum filling) and then sealing the filling holes (2022) using a UV or epoxy glue (2025).

A lens according to an embodiment may also comprise a sensor or transducer element (2007) to detect information concerning the required focal state from the eye. For example, this may be a small inductive coil that is sufficiently sensitive to detect the electrical signals sent to the ciliary muscles of the eye to trigger a change to the refractive power of the lens. Such an inductive coil can be integrated onto one or more substrates of the lens. The coil is not required to provide an accurate absolute measurement of a parameter associated with muscle activity. Instead, it is sufficient that it can distinguish between two different states, associated with the eye attempting to focus on a near or a far object. As such, it is a relative measurement that is required, which reduces the accuracy required of the sensor (2007). Means may also be provided for calibrating the lens to a particular user, through a calibration procedure, where the signals are detected when the user deliberately focuses at distant and near objects, and records whether the focus is set to the correct level on the control system.

Alternatively, the sensor may use positional sensors to detect the relative position of a lens with which it is paired, compare it to a reference position, and switch the focal power of the lens accordingly. Alternatively, the sensor may detect the position of the lens relative to the primary gaze position and switch the focal power of the lens accordingly. Alternatively, the sensor may detect the downward movement of the lens relative to its customary position and switch the focal power of the lens accordingly.

Alternatively, sensor (2007) may be configured to detect a brightness level, so that the user can initiate a change in focal state by blinking both eyes or, preferably, only one eye. The sensor (2007) may be a phototransistor or photodiode, which is sensitive to the ambient light level to which it is exposed. In order to prevent inadvertent switching of the focal state, which may be caused by a gradual change in lighting, which would be experienced quite commonly in day to day use, the microcontroller (2005) may monitor the signal provided by the sensor (2007) and trigger a change in focal state, only if there is a sudden momentary change in brightness which satisfies certain thresholds. For instance, the light level should drop to below a threshold associated with the user's eyes being closed for more than a certain predefined period. This ensures that a change in focal state is not triggered by normal blinking, which lasts for, typically, 300-400 ms. If a period of, for example 800 ms is specified, then this requires the user to consciously close their eyes for a longer period than that associated with blinking. A further threshold could be introduced so that if the user closes their eyes for a period in excess of, for example, 2 seconds, then this is not interpreted as an instruction to change focal state. This would allow the user to close his/her eyes for a short period without changing the focal state of the lenses.

Obviously, in normal use, the ambient light level changes frequently and both eyes are typically exposed to the same ambient level. In a further embodiment, the lenses may be configured such that the user is required to wink (close only one eye) for a predefined period in order to change focal state of both lenses. In this way, the chance of false triggering may be reduced, since the light level experienced by both eyes can be compared and switching is only triggered if a wink, rather than a blink is detected. It is unlikely that the left and right eyes are usually exposed to different ambient light levels.

FIG. 8 illustrates the various thresholds associated with using a blink or a wink to trigger a change in focal state. The vertical axis represents the light level detected by sensor (2007) and reported to microcontroller (2005). The horizontal axis represents time. Once the measured light level (8040) drops below a threshold (8000), a timer is started at time 8010 and if the light level remains below the threshold (8000) until a first time threshold 8020 is reached, then a switch in focal state is triggered. In other words, if a user keeps one or both eyes (as required) closed for a period 8015, then focal state is switched.

Also shown is a second time threshold (8030). If the measured light level remains below the threshold (8000) for a longer time, as set by this threshold (8030), then no switch in focal state is triggered, allowing the user to close their eyes for a period without switching focal state. In effect, too short a wink or a blink will not trigger a change in focal state, neither will too long a wink or a blink. The wink or blink, once started, must end in the time period falling between thresholds 8020 and 8030 to trigger a change in focal state.

The change in focal state is akin to a bistable switch. The trigger signal causes it to change state from whichever state it is currently in. In this way, the user simply repeats the wink or blink action to change from one state to another.

In its simplest mode, a single lens (3001) may be provided for use by a person. However, in most practical circumstances, a user will use a pair of lenses and so it is important that the pair of lenses are properly configured to allow them to operate in unison. It is also important that the focal state of other nearby lenses that are not being used by the same person are not interfered with, and that the system recognises the paired lenses associated with the same user.

Alternatively, the sensor may be incorporated into an external control unit, and not the lenses themselves. For example, the unit may include a camera and software that detect the eyes of the user and determines their relative direction of gaze. Such eye-tracking software will enable the control unit to calculate whether or not the eyes are focussed on near or far objects by their relative position and orientation. Switching of the lens to the appropriate state may then be done using RF, or alternatively an IR signal that can be detected by the lens.

The means by which the lens or lenses is set up for use and the signalling required in use will now be described.

There are two basic modes by which embodiments of the present invention can operate—a standalone mode or a controlled mode. In each mode, the lens may be programmed using a separate, suitably equipped and programmed computer device, such as a mobile phone, smart watch or wristband, Bluetooth headphones or jewellery, laptop computer or other computer device. However, in the standalone mode, the lens or lenses store the necessary commands and operate independently of an external controller. In the controlled mode, illustrated in FIG. 3, an external controller (3003) i.e. one that is external to the lens or lenses (3001), is provided within a separate computer device, such as a dedicated controller, or as part of a smart device (3004) such as a smart watch, mobile phone, etc.

In standalone mode, the lens or lenses, once in situ, operate entirely independently of any external controller. This requires the lens or lenses to store enough power for the expected duration of their use i.e. normal waking hours for a typical user, such as 7 am to 11 pm, for instance.

In controlled mode, the lens or lenses receive control signals from an external controller (3003), which provides the necessary control signals that cause the lens or lenses to switch focal state. Advantageously, as well as providing control signals, the use of an external controller allows power to be supplied to the lens or lenses also, which facilitates the use of a relatively smaller power source on the lens.

The micro-controller (1005, 2005) for each lens may be programmed to have a unique identification code (e.g. an RFID code), and that code can be read by another lens, or by the system controller (3003). Where a further processing device is used, the computer (3004) communicates (3005) with the controller (3003) using conventional communication methods, such as Low Power RF (LPRF, for example in the ISM band), Bluetooth, Low energy Bluetooth (LBE) or wi-fi. Alternatively, communication may be made using infrared to trigger, and potentially to power, the lenses. Communication between the controller (3003) and the paired lenses (3001) is achieved using a radio signal of limited range, typically in the region of 1-2 m (3006). Two way communication between lens and controller is only acted upon by the controller if the signal is received from the paired lenses (3001) and not from other lenses (3002) that may be in proximity, since these other lenses may be another pair of the user's lenses which are not in-situ, or may even belong to another person altogether.

This ensures that only the intended lenses are instructed to change focal state, since issuing an instruction to a non-paired lens or lenses is clearly undesirable.

Regardless of whether they are operating in standalone or controlled mode, as set out above, embodiments of the invention have three primary functional modes: a setup mode, an operation mode and a charging mode.

The set-up mode comprises the steps:

A lens (3001) is paired with the corresponding controller unit (3003) by sending a signal from the controller unit to the lens. The controller unit is programmed to recognise the unique identifier of each lens and links the two lenses together. This pairing is usually done when a new lens pair is required, for example at the beginning of a day, week or month. It may also be done, when a new lens is introduced to the lens pair, for example, if only one lens is replaced part way through the usual cycle due to loss or damage. Each lens is provided with a unique identifier at the time of manufacture, to ensure that pairing and communication, in use, occurs only between recognised and associated devices, as required.

The pairing process is illustrated in FIG. 4, which also shows the message exchange from the controller (3003) and the lenses (3001a, 3001b). The controller is required in order to create a link between the pair of lenses, which are typically not connected beforehand. The controller, possibly via a User Interface provided by the further smart phone or similar computer device (3004), scans (4000) an identifier associated with the first lens (3001a). The identifier may be provided in the form of a bar code, QR code or similar, on the packaging of the lens (3001), or it may be in the form of an RFID tag integrated into the lens, which requires the controller to be brought into close proximity with the selected lens. In this case, care may be needed to ensure that the correct lens is scanned.

Once the ID of the lens (3001a) has been captured by the scanning process (4000), the controller sends a message (4010) to the lens (3001a) to associate the controller with the specific lens (3001a). The lens replies to the controller with a handshake/confirmation message (4020). From this point, the lens (3001a) is paired with the controller (3003) and will only accept control messages from it, and not another controller.

The second stage of the paring process, is to pair the controller (3003) with the second lens (3001b), if present. The process is substantially identical with that just described for the first lens, whereby the controller (3003) physically scans (4030) an identifier for the second lens (3001b), sends an association message (4040) to the second lens (3001b) and receives a handshake/confirmation message (4050).

If the user has set up a pair of lenses (as opposed to a single lens), then if the lenses are to operate in standalone mode, without an external controller required for normal use, then the two lenses need to be made aware of each other's identities. This process is shown in FIG. 5.

The controller (3003) sends a message (5000) to the first lens (3001a) informing it of the identity of the second lens (3001b). The first lens responds to the message with a confirmation (5010), indicating that it now knows the identity of the second lens (3001b). The controller then sends a message (5020) to the second lens (3001b) informing it of the identity of the first lens (3001a). The second lens responds with a confirmation (5030). Now, each lens is aware of the unique identity of the other with which it must operate in normal use. In order to check that the first and second lenses can communicate properly, the first lens (3001a) sends a message (5040) to the second lens (3001b). The second lens (3001b) responds with a confirmation message (5050) back to the first lens (3001a), and the first lens sends a message (5060) to the controller indicating that 2-way communication has been established between the first and second lenses. The first lens (3001a) is designated as the master lens in this scenario. The controller (3003) is then not required for normal use. The designation of a master lens may correspond to a left or right lens, as required, and allows one of a pair of lenses to be the primary (or only) lens to communicate with the controller.

Once paired in this way, the user may wear the lenses in the usual way.

In an alternative embodiment, the lenses (3001a, 3001b) may be charged in a charging station, which is aware of the identities of the lenses, by use of an RFID tag in each lens. The controller (3003) may communicate with the charging station such that the lenses included in the charging station only are paired together and/or with the controller.

The pairing process may comprise a synchronisation step between the control unit and the lens. On pairing, the clock of the lens will not be synchronised with the control unit. Even if this is not the first time a lens has been paired (e.g. each morning on the lens being used) the synchronisation of the lens may not coincide with the control unit. A synchronisation correction signal may be incorporated into the pairing sequence.

The setup mode may further comprise a calibration step, where the user focuses on a particular focal plane (either near to, or far from the eye) and the status of the two lenses is checked. This may be a check of the signal from the ciliary body (via sensor 2007), whereby different measurements from the inductive coil are recorded when focussing near or far are recorded, a reading of the relative position of the two lenses, or some other form of eye-tracking (for example, from visual recognition of the facial system through a camera in communication with the controller system). Eye-tracking systems are well known in the art and may be configured to detect the relative spacing between a user's pupils and on the basis of changes in the spacing if focused on a near or a far object, calibration may be performed.

The charge in the lens or lenses may be detected and information sent to the communicating computer device (3004), for example through the controller (3003). This is to ensure that the lenses are suitably charged for a defined period of use, typically a day. If insufficient charge is present, the computer device or controller may issue a warning to the user who may be prompted to initiate a charge process (see below). If the pairing process has just been completed, the lenses will usually have been charging overnight and should be sufficiently charged for the day ahead.

Each lens may be in one of a plurality of power modes, depending on its status. In a low power mode, the lens is inactive. Such a mode may require no power (for example, where pairing is done by reading a barcode or RFID on the lens or its storage unit) or it may have a very low power associated with the lens occasionally waking up (e.g. once per 20 seconds) to listen for a pairing sequence from a control unit). Following pairing with the control unit, the lens becomes active and is therefore in a higher power mode. In this mode, the lens will listen for instructions on a short time scale, typically of between 20 ms and 200 ms, but preferably 100 ms. These instructions will activate or deactivate the lens to set it to the required focal state. On each operation, the lens will send a receipt of the instruction to the control unit, together with the lens ID code. If the control unit does not receive that receipt from the correct lens within a short time (e.g. 300 ms, or 3-5 operating cycles of the lens clock) it will repeat the attempt immediately.

The lens may have an OFF power mode, set by the manufacturer. In this instance, the lens uses no power at all, not even to listen for charging or pairing instructions from a controller unit or charging unit. In this instance, switching to the low power mode is done by external power provided by the controller or charger, that activates the lens. The lens is then switched to the low power mode, where it waits to receive the pairing instruction described above. Finally, the lens is switched into active mode after pairing and charging. It would be intended that the lens is switched into the lower power mode from the OFF mode just once in the lenses usable lifetime. However, in practice this would also be the means by which the lens can be reactivated after being completely discharged.

The lens clock may be resynchronised during the charging and pairing operations in the low-power mode, and regularly throughout the high-power operating mode.

In the operating mode, the steps differ, depending upon whether the lenses are operating in controlled or standalone mode. In controlled mode, the normal steps are as shown in FIG. 6, and as follows:
1. The external controller (3003) determines a change in focal power is required. This may be triggered by the user, or automatically by the external controller;
2. A signal is sent to both lenses, from the controller (3003) that includes the unique identification codes for the two lenses. Firstly a message (6000) is sent to the first lens (3001a), which acknowledges with a handshake (6010) to indicate safe receipt. Then, a message (6020) is sent to the second lens (3001b), which acknowledges with a handshake (6030)
3. If handshake signals (6010, 6030) are received from both lenses, a trigger signal is sent to the lenses by the controller. The signal is sent from the controller to each of the paired lenses in sequence (6040, 6050), meaning that each lens switches focal state at a slightly different time. The signalling is conducted such that the time difference is imperceptible to the user.
4. Once the lenses receive the trigger signal, the microcontroller sends an appropriate signal to the electrodes to cause the change in focal state to occur.

Steps 1 to 4 typically take less than 100 ms to complete, to ensure that the focal change is made as required, at a rate which is pleasing or imperceptible to the user.

In an alternative embodiment, step 2 above may be repeated on a regular basis, and then step 1 is immediately followed by step 3. This ensures that the controller is in constant contact with the lenses and removes the need to check contact status immediately prior to instructing a change in focal state. This mode of operation requires more power, but the change in focus is more immediate, and the user is less likely to notice any delay or lag. In practical situations, the availability and capacity of the on-lens power storage may dictate which option is selected, as there will tend to be a compromise between speed of operation and power consumption.

Means may be provided where the system remains in the stand-alone or constant contact mode for a pre-set time duration before changing back to the lower power standard operating mode. This is because certain tasks may require regular switching from one focal length to another, for which the fastest response is necessary. The duration of this pre-set period may be set in the software of the operating system according to the user's preferences, or may be factory set.

In standalone mode, the steps are similar, except the initial decision to change focal state is derived from the lens (3001a) itself rather than from the external controller. The trigger for this is derived from sensor (2007), and the steps are as shown in FIG. 7 and as follows:

1. The first lens (the primary lens) (3001a) registers via sensor (2007) a need to change focal state. It then sends a message (7000) to the second lens (3001b) including its unique identifier, to ensure that the second lens is functioning correctly.
2. The second lens (3001b) responds with a handshake (7010) to acknowledge it has received the message.
3. The first lens then sends a trigger message (7020) to the second lens (3001b) so that its microcontroller sends an appropriate signal to the electrodes to cause the change in focal state to occur. The first lens (3001a) may either trigger a change in its own focal state immediately before or after sending the trigger message to the second lens (3001b), with the overall effect being one of near-simultaneous change in the focal state of both lenses.

As with the controlled-mode described previously, the first and second lens may be in regular contact, so that the need for the first lens to signal the second lens and await a handshake is avoided and a trigger signal may instead be sent as soon as a need to change focal state is detected.

The controller in the standalone mode is incorporated into one lens. It may alternatively be incorporated into both lenses and, during the pairing process, one of the two controllers is declared by the user to be the master controller and the other a slave controller. The controller in the standalone mode (i.e. the master controller) may receive trigger signals from only the sensor in the lens within which it is comprised, or from both lenses, wherein a trigger event from the slave lens is detected and communicated to the master lens. The controller may be set to respond either to a single trigger event, or to trigger events from both lenses, received within a certain time period (e.g. 500 ms).

In the charging mode, a high power radio signal is sent to the lenses, which is then used to charge the charge storage unit (1006, 2006) rather than power the lenses. Charging mode is entered by placing the lens or lenses into a receptacle once the user has removed it or them. The charging process is performed wirelessly, in the sense that no physical connection is required, and power is transferred from the charging device to the on-lens battery or similar charge source by means of RF energy, using well known principles, used commonly in charging electric toothbrushes and the like. Alternatively, other energy sources might be used. For example, if triggering is done by a separate control unit via an IR signal, then charge may also be supplied through the Photosensor for the IR when in charging mode, rather than operation mode.

In all embodiments, the lens is usually operated such that one of the commonly required focal lengths requires no power to the electro-refractive medium. Often, the lens will be arranged to operate for long distance vision without an operating voltage applied. In this fashion, the charge from the charge storage unit can be conserved, and only used when in the near vision (i.e. short focal length) state. This is predicated on the understanding that for most users, the lens would normally be used for distance vision and more occasionally used for near vision activities, such as reading or computer work. However, lenses with the opposite configuration may also be used.

The control system (3003) is operable to read the electrical signals to both eyes, and only responds with a trigger signal when the signals are from both eyes within a time delay that is suitably short to indicate that a change in focus is required. This ensures that unnecessary changes are not triggered as a result of a signal from a single lens only, which would be annoying to the user.

In one embodiment, referred to as controlled mode, power (or charge) is stored on the lens for operating for only a period of several minutes to an hour. In such instances, charging may be performed by manually bringing the separate controller unit (3003) to the vicinity of the eyes. For example, the controller may be mounted within a wristwatch or bracelet. In such a system, the triggering of the change in focal state, may be manual—in the sense that the need to change focal state is recognised and triggered by the user—and the lens can operate without comprising the microcontroller (1005, 2005), sensor (2007), and/or transceiver (1004, 2004).

In such instances, the power necessary to cause a high voltage to change the focal state of the lens is triggered by the power supplied wirelessly from the separate controller unit (3003). Once triggered, the lens is re-charged for the remainder of the time that the controller is in proximity to the lenses.

The lenses may be triggered to change focal state by an eye tracking and a camera system, as described previously. Eye tracking systems are used in systems which are required to display different information to different viewers, such as in 3D displays and multiviewer displays. Such systems are able to distinguish two eyes that are focused onto the system display (in proximity to the camera) and cause switching when the eyes are both directed towards the camera.

In a further embodiment, the camera may detect the direction of the eyes and calculate whether or not the relative angle between the eyes is focused on the short or far distance (regardless of whether it is towards the camera or not). This provides another way in which a decision can be taken on whether to trigger a change in focal state or not. The set up procedure for such a system might include calibration of the individual user through software, wherein the user sets the eye separation relative to the orientation of the face for short distance and long distance viewing.

In another embodiment, either the internal microcontroller or external controller is operable to receive signals from the sensors (2007) of the paired lenses that indicate a change in their relative separation and/or orientation. If the change is within a certain range of values, then the change in focal strength is triggered. Typically, the change of spacing between paired lenses will be between 2 mm and 4 mm. For example, this can be done using eye tracking software from an external camera mounted (for example) on the users wrist. This algorithm uses face recognition techniques to determine the relative orientation of the face to the camera, and then determine the orientation of the eyes and their relative spacing.

Alternatively, the contact lenses may incorporate a grating tuned to reflect an infrared signal emitted by the controller device. A detector on the controller then tracks reflections from the front of the cornea (the mirror on the contact lens) and the rear of the eye through the pupil. Where no reflection from the pupil is detected, the controller uses the relative position of the mirrors to calculate the relative separation and orientation of the eye pair.

In another embodiment, the focal strength of each lens is adjusted during the calibration process, whereby the user indicates the level of focal strength adjustment that gives the best focus for different viewing conditions (for example, reading or distance). In such cases, the voltage level required for such a focal strength is also communicated to the lens together with its identification code. In this way, each lens may receive different signals to give different focuses.

It is possible that in a pair of lenses, each lens will have a different focal power, which requires the user to ensure that the correct lens is worn in the correct eye. Upon wearing the lenses, after pairing, the lenses may cycle though their focal states individually in a known pattern so that the user can be sure that the correct lens is in the correct eye. For instance, the right eye may switch between ‘near’ and ‘far’ modes and then a few seconds later, the left eye may do the same. The user will be aware of the order—right followed by left—and can swap the lenses in case they have been inserted incorrectly.

In the foregoing description, when a series of steps in a method are presented, it may be possible to deviate from the explicit order given, without departing from the scope of the invention, which is defined by the appended claims. In other words, a given order is not to be regarded as limiting and changes in the order are envisaged, which do not change the scope of the invention.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A dynamically switchable contact lens, operable to switch between first and second focal states, each focal state having a different optical property, the lens comprising:
a power source;
a controller, operable to control the operation of the lens;
a transceiver operable to communicate with a second lens and/or an external controller;
wherein the controller is operable to pair the lens with the second lens by means of the external controller.

2. The lens of claim 1 comprising a liquid crystal material, disposed in a cavity and wherein the optical property of the lens is altered according an electrical field applied to the liquid crystal material.

3. A dynamically switchable contact lens as claimed in claim 1 wherein the controller is operable to perform the steps of:
(a) scanning, using the external controller, an identifier associated with the first lens;
(b) transmitting to the first lens an association message to associate the external controller with the first lens;
(c) scanning, using the external controller, an identifier associated with the second lens; and
(d) transmitting to the second lens an association message to associate the external controller with the second lens.

4. A method of associating first and second dynamically switchable contact lenses with an external controller, comprising the steps of:
(a) scanning, using the external controller, an identifier associated with the first lens;
(b) transmitting to the first lens an association message to associate the external controller with the first lens;
(c) scanning, using the external controller, an identifier associated with the second lens; and
(d) transmitting to the second lens an association message to associate the external controller with the second lens.

5. The method of claim 4 further comprising pairing the first and second lens, such that they are later operable in a standalone mode, without the external controller, the pairing comprising the steps of:
(e) the external controller transmitting a message to the first lens, including second lens identity information;
(f) the external controller transmitting a message to the second lens, including first lens identity information;
(g) the first lens transmitting a message to the second lens;
(h) the second lens transmitting a message to the first lens in response; and
(i) the first lens transmitting a message to the external controller to indicate that standalone mode is operable.

6. The method of claim 5 further comprising the steps of the first and second lens, respectively, transmitting confirmation messages to the external controller after steps (e) and (f).

7. A method of changing focal state of first and second dynamically switchable contact lens, comprising the steps of:
(a) determining that a change of focal state is required;
(b) transmitting from an external controller a message to the first lens, including first lens identity information;
(c) receiving at the external controller a response message from the first lens;
(d) transmitting from an external controller a message to the second lens, including second lens identity information;
(e) receiving at the external controller a response message from the second lens;
(f) transmitting to the first lens a trigger signal to change focal state; and
(g) transmitting to the second lens a trigger signal to change focal state.

8. The method of claim 7 wherein the step of determining that a change of focal state is required is triggered either by a user or by the external controller.

9. The method of claim 7 wherein steps (b) to (e) are repeated on a regular basis such that if it is determined that a change of focal state is required, steps (f) and (g) may be completed immediately after step (a).

10. A method of changing a focal state of first and second lenses, operating in a standalone mode, comprising the steps of:
(a) the first lens determining that a change in focal state is required;
(b) the first lens transmitting a message including second lens identity information to the second lens;
(c) the second lens transmitting a message to the first lens in response;
(d) the first lens triggering a change in its own focal state;
(e) the first lens transmitting a trigger message to the second lens to change focal state; and
(f) the second lens triggering a change in its own focal state.

11. The method of claim 10 wherein the step of the first lens determining that a change in focal state is required is determined on the basis of a ciliary muscle sensor or by means of a detector operable to sense a duration of a user's wink or blink and to determine that a change in focal state is required if the wink or blink is of a defined duration.

12. The method of claim 11 wherein the defined duration is longer than a predefined minimum time and shorter than a predefined maximum time.
